(19) United States
(12) Patent Application Publication
(10) Pub. No.: US 2011/0015694 A1
(43) Pub. Date: Jan. 20, 2011

(54) PREFABRICATED HEADER FOR HERMETICALLY SEALED DEVICE

(75) Inventors: William Alexander, Tualatin, OR (US); Christopher Fleck, Marina del Rey, CA (US)

Correspondence Address: PACESETTER, INC., 15900 VALLEY VIEW COURT, SYLMAR, CA, 91392-9221, US

(73) Assignee: PACESETTER, INC., Sylmar, CA (US)

(21) Appl. No.: 12/504,113

(22) Filed: Jul. 16, 2009

(51) Int. Cl. A61N 1/375

(52) U.S. Cl. 607/36

(57) ABSTRACT

A prefabricated header assembly is hermetically welded to a housing of a device. The header assembly includes a metal base and a circuit embedded in an encapsulating material. The metal base includes a hermetically sealed feedthrough for providing an electrical connection between one or more components located in the housing and the circuit of the header assembly. During assembly, the metal base is placed over an aperture defined in the housing such that the metal base may be welded to the housing to complete the hermetic sealing of the device. In some implementations the header assembly also includes a battery assembly.

### TECHNICAL FIELD

This application relates generally to hermetically sealed devices and, more specifically, but not exclusively to a prefabricated header for a hermetically sealed device.

### BACKGROUND

Conventional implantable devices are sealed to prevent fluid and tissue from entering the devices. For example, in an implantable device constructed of a multi-section metal housing, the sections of the housing may be hermetically welded to hold the sections together and seal the device.

Some types of implantable devices connect to other implantable circuits. For example, an implantable cardiac device provides stimulation signals to and/or receives cardiac signals from one or more implantable cardiac leads. Accordingly, an implantable device may include a mechanism such as a header that provides connectivity to an external circuit. The housing of such an implantable device may include a hermetically sealed feedthrough for one or more electrical conductors. One side of this feedthrough is connected to a circuit located within the housing. After this connection is made, the housing is sealed.

The other side of the feedthrough is connected to a circuit (e.g., a connector) of the header. In some cases such a header may be constructed using a cast-in-place process. For example, a mold may be placed around the header circuit and an epoxy injected into the mold. Once cured, the epoxy forms a header body which is affixed to the housing.

In practice, a cast-in-place process may not be a particularly efficient manufacturing process. For example, a cast-in-place process is relatively complicated and generally involves the use of skilled labor. In particular, the epoxy should be properly mixed, the surfaces of the housing should be properly prepared, and strict preparation and curing times should be observed to facilitate adequate epoxy adhesion to the housing and provide a housing with sufficient structural integrity. Such labor dependent processes may, however, negatively affect the manufacturing yield. For example, when a header is not properly formed on the housing, the entire implantable device may need to be put through an extensive rework process. Also, a cast-in-place process typically has a relatively long cycle time and the equipment used in such a process may consume a relatively large amount of a manufacturing room floor. In view of the above, a need exists for more effective techniques for manufacturing implantable devices and associated components.

### SUMMARY

A summary of several sample aspects of the disclosure and embodiments of an apparatus constructed or a method practiced according to the teaching herein follows. For convenience, one or more aspects or embodiments of the disclosure may be referred to herein simply as “some aspects” or “some embodiments.”

The disclosure relates in some aspects to a header assembly that is hermetically welded to a housing of a device such as an implantable device. For example, a header assembly may include a metal base and a circuit embedded in an encapsulating material that is attached (e.g., adhered) to the metal base. The metal base is placed over an aperture defined in a housing of the device such that the metal base may be welded to the housing to complete the hermetic sealing of the device.

The metal base includes at least one hermetically sealed feedthrough for providing an electrical connection between one or more components located in the housing and the circuit of the header assembly. Once the metal base is welded to the housing, the feedthrough thereby provides connectivity between the internal and external circuits of the device, while maintaining a hermetical seal.

The header assembly may be prefabricated in some aspects to simply the manufacturing process for the device. For example, prior to assembly of the device, the connections between the header circuit and one side of the feedthrough may be made. In addition, the encapsulating material may be formed over the header circuit and the base. Consequently, assembly of the device may simply involve connecting the other side of the feedthrough to the internal component(s) of the device and welding the base to the housing of the device.

In some implementations the header assembly includes a battery assembly. For example, a battery assembly that is to be installed within the housing may be mechanically coupled to the header assembly via a coupling member that passes through the aperture of the housing. In some implementations this mechanical coupling may be accomplished by a bracket or other suitable mechanism attached to the base of the header assembly.

In some aspects, through the use of these and other techniques as taught herein, a device such as an implantable device may be manufactured in a more efficient manner. For example, the use of a welding (e.g., laser welding) process to attach a header assembly to a device may result in higher yields (e.g., less scrap or rework) than conventional techniques. Such a process may be automated (e.g., using a robotic welder). In addition, it may be easier to verify and inspect the quality of such an attachment (e.g., the quality of a weld may be easily inspected). Also, the manufacturing time of such an operation may be shorter than the manufacturing times associated with conventional techniques (e.g., which may involve relatively long cure times for the header material).

### BRIEF DESCRIPTION OF THE DRAWINGS

These and other features, aspects, and advantages will be more fully understood when considered with respect to the following detailed description, the appended claims, and the accompanying drawings, wherein:

FIG. 1A is a simplified diagram illustrating a housing and a header assembly of an embodiment of an implantable device;

FIG. 1B is a simplified diagram illustrating how the header assembly and the housing of FIG. 1A may be assembled;

FIGS. 2A and 2B are simplified diagrams illustrating an embodiment of electrical connections for a header assembly;

FIG. 3 is a simplified diagram illustrating sample components of an embodiment of a header assembly;

FIGS. 4A and 4B are simplified diagrams illustrating an embodiment of an anchor member for a header assembly;

FIG. 5 is a simplified diagram illustrating an embodiment of a circuit for a header assembly;

FIG. 6A is a simplified diagram illustrating a header assembly including a battery assembly that is configured to be placed within a housing;

FIG. 6B is a simplified diagram illustrating the header assembly of FIG. 6A where the battery assembly is placed within a bottom portion of the housing;

FIG. 7 is a simplified diagram illustrating another embodiment of a header assembly and housing; and

FIG. 8 is a simplified flowchart of an embodiment of operations that may be performed to assemble a hermetically sealed device.

In accordance with common practice the various features illustrated in the drawings may not be drawn to scale. Accordingly, the dimensions of the various features may be arbitrarily expanded or reduced for clarity. In addition, some of the drawings may be simplified for clarity. Thus, the drawings may not depict all of the components of a given apparatus (e.g., device) or method. Finally, like reference numerals may be used to denote like features throughout the specification and figures.

### DETAILED DESCRIPTION

The description that follows sets forth one or more illustrative embodiments. It will be apparent that the teachings herein may be embodied in a wide variety of forms, some of which may appear to be quite different from those of the disclosed embodiments. Consequently, the specific structural and functional details disclosed herein are merely representative and do not limit the scope of the disclosure. For example, based on the teachings herein one skilled in the art should appreciate that the various structural and functional details disclosed herein may be incorporated in an embodiment independently of any other structural or functional details. Thus, an apparatus may be implemented or a method practiced using any number of the structural or functional details set forth in any disclosed embodiment(s). Also, an apparatus may be implemented or a method practiced using other structural or functional details in addition to or other than the structural or functional details set forth in any disclosed embodiment(s).

The disclosure relates in some aspects to a header assembly that may be hermetically welded to a housing of a device, where the header assembly includes a hermetically sealed feedthrough for connecting circuitry in the header assembly to circuitry located within the housing. For illustrations purposes, these and other aspects of the disclosure will be described in the context of a header assembly for an implantable medical device (e.g., a pacemaker, an implantable cardioverter defibrillator, an implantable stimulation device, an implantable monitoring device, and so on). It should be appreciated, however, that the teachings herein may be applicable to other types of devices (e.g., devices that are not implanted).

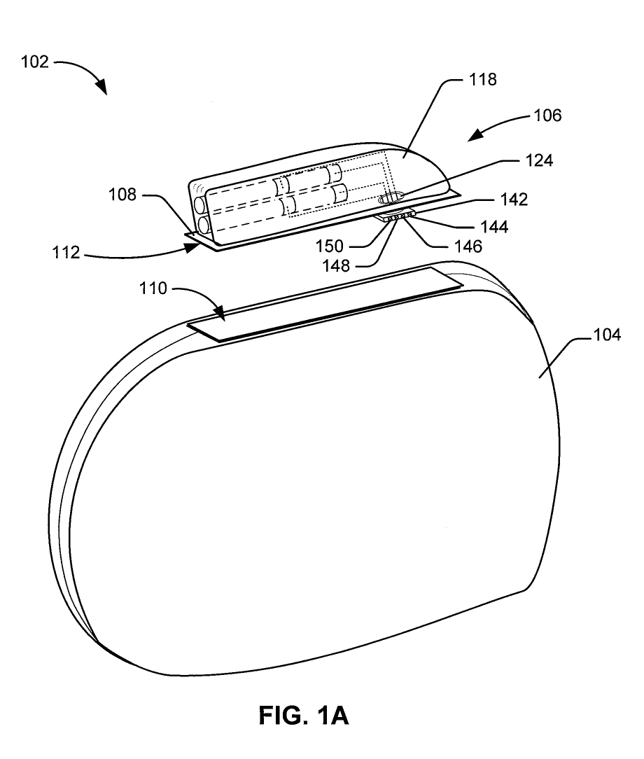

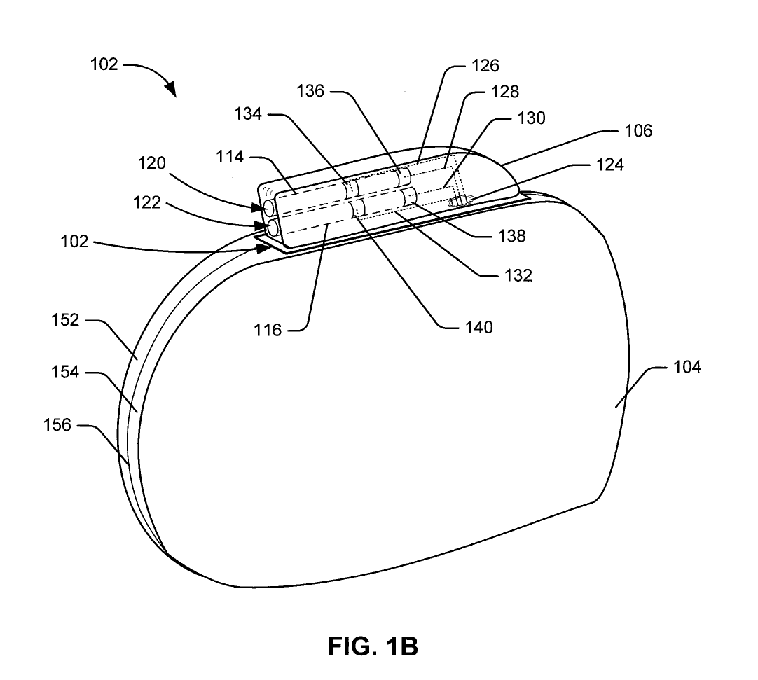

FIGS. 1A and 1B depict an embodiment of implantable device 102 that includes a housing 104 and a header assembly 106. As illustrated in FIG. 1A, the header assembly 106 may be a prefabricated assembly that includes a metal base 108. The base 108 is configured (e.g., sized and shaped) to be installed over an aperture 110 defined by the housing 104. Once installed, the edges of the base 108 (e.g., edge 112) are hermetically welded to the housing 104 (as shown in FIG. 1B).

The header assembly 106 includes at least one circuit (e.g., connectors 114 and 116 in FIG. 1B) that is embedded in an encapsulant 118. The encapsulant 118 insulates the circuit components from one another and may also hold these components in place. The connectors 114 and 116 are embedded in the encapsulant 118 in a manner that enables implantable leads (not shown) to be inserted into openings 120 and 122 associated with the connectors as indicated in FIG. 1B.

The base 108 includes a hermetically sealed feedthrough 124 for one or more conductors. As shown in FIG. 1B, the feedthrough 124 includes four conductors 126, 128, 130, and 132, each of which connects to a respective contact 134, 136, 138, and 140 of the connectors 114 and 116 (in this example two connectors are shown; other implementations may use a different number of connectors). As shown in FIG. 1A, the conductors 126, 128, 130, and 132 also are connected to contacts 144, 146, 148, and 150 of a connector 142 that is mounted on the bottom of the base 108. Thus, the feedthrough 124 enables the connectors 114 and 116 to be coupled to one or more circuits (not shown in FIGS. 1A and 1B) that are installed within the housing 104, while maintaining a hermetic seal for the housing 104.

As will be described in more detail below, the housing 104 may be constructed of subcomponents (bottom portion 152 and top portion 154 in the example of FIG. 1B). Such a construction may facilitate installing circuitry in the housing 104 and connecting the feedthrough 124 to contacts associated with this circuitry. Here, once the header assembly 106 is in place, the bottom and top portions 152 and 154 may be hermetically welded together (e.g., at a seam 156) in conjunction with welding the base 108 of the header assembly 106 to the housing 104.

The components of a device as taught herein may be constructed of various materials. For example, for implantable devices, the housing 104 and the base 108 may be made of a biocompatible metal such as titanium, MP35N, or some other suitable material. In applications where the device is not implantable but is to be hermetically sealed, the housing and the base may be made of a material (e.g., a metal) that may be hermetically sealed (e.g., laser welded). Other materials may be used in other applications.

Various materials also may be used for the encapsulant. For example, the encapsulant may be a plastic, a thermoplastic, a two-part resin, an epoxy, a rigid silicone-based plastic (or thermoplastic), urethanes such as Elast-Eon™ by AorTech International PLC, or some other material that is suitable for a designated application. Also, the encapsulant may be formed over the header components in various ways. For example, epoxy casting, overmolding, injection molding, reaction injection molding, or some other suitable process may be employed.

In addition, various techniques may be employed to hermetically weld the metal components together. For example, techniques such as laser welding, ultrasonic welding, resistance welding, or some other type of welding may be employed in different embodiments.

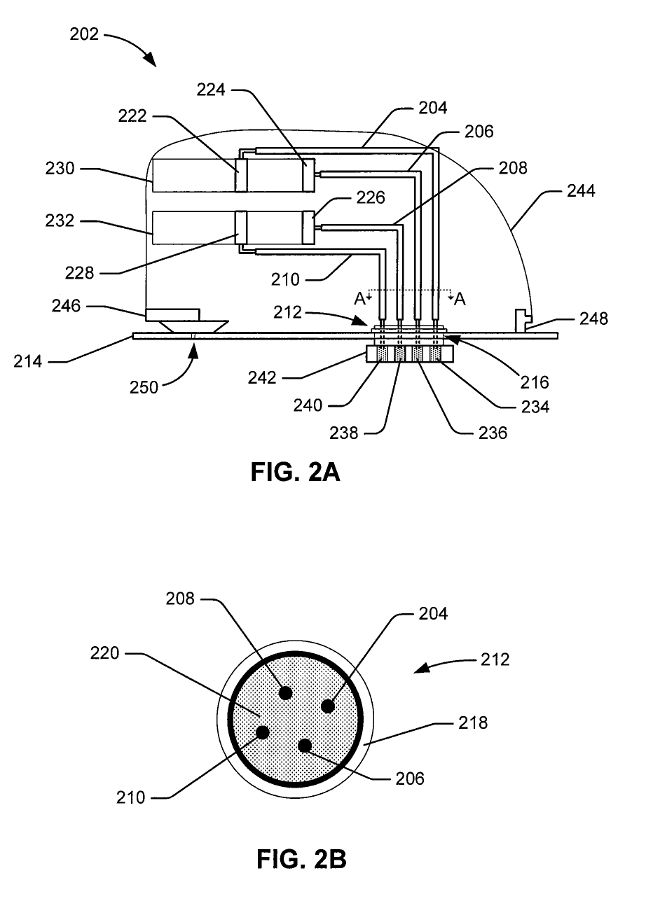

FIGS. 2A and 2B illustrate, in a simplified manner, how the circuitry of a header assembly 202 may be connected to conductors 204, 206, 208, and 210 of a hermetically sealed feedthrough 212. It should be appreciated that the components of FIGS. 2A and 2B may correspond to similarly named components of FIGS. 1A and 1B. For illustration purposes, the example of FIGS. 2A and 2B depicts the header assembly 202 without an encapsulant. Thus, these figures represent in some aspects how a header assembly may be constructed prior to the encapsulation process.

FIG. 2A illustrates how the feedthrough 212 may be incorporated into a base 214 of the header assembly 202. Here, the feedthrough 212 is inserted into a hole 216 of the base 214 (as represented by dashed lines through the base 214). The view of FIG. 2B (which corresponds to the view A-A of FIG. 2A) depicts an outer edge 218 of the feedthrough 212 than may be hermetically welded to the base 214.

FIGS. 2A and 2B also illustrate how the conductors 204, 206, 208, and 210 pass through the feedthrough 212. As shown in FIG. 2B, the conductors 204, 206, 208, and 210 are embedded in an insulating material 220 (e.g., a ceramic material) of the feedthrough 212. As shown in FIG. 2A, top portions of the conductors 204, 206, 208, and 210 are connected to respective contacts 222, 224, 226, and 228 of connectors 230 and 232. In addition, in this example bottom portions of the conductors 204, 206, 208, and 210 are connected to respective contacts 234, 236, 238, and 240 of a connector 242.

FIG. 2A further illustrates that in some embodiments the header assembly 202 may include other members (e.g., an antenna 244 and/or anchors 246 and 248) that improve the structural integrity and/or functionality of the header assembly 202. For example, after an encapsulant (not shown) is molded over the components of FIG. 2A, the antenna 244 and the anchors 246 and 248 may serve to mechanically hold the encapsulant and the base 214 together and provide RF functionality. In some implementations the anchor 206 may comprise a coupling that couples the antenna to a conductor (not shown) that passes through a feedthrough 250 in the base 214.

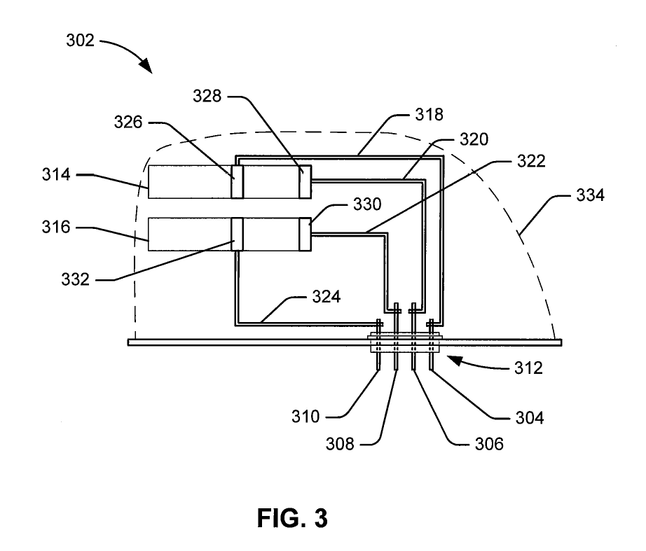

FIG. 3 illustrates another simplified example of how conductors 304, 306, 308, and 310 of a feedthrough 312 may be connected to a pair of connectors 314 and 316 of a header assembly 302. In this case, the conductors 304, 306, 308, and 310 comprise short wires that extend from the bottom and the top of the feedthrough 312. Conductors 318, 320, 322, and 324 are connected (e.g., wire bonded, welded, soldered, etc.) to top portions of respective ones of the conductors 304, 306, 308, and 310 as shown. The conductors 318 and 320, are connected (e.g., welded, soldered, etc.) to respective contacts 326 and 328 of the connector 314. Also, the conductors 322 and 324 are connected (e.g., welded, soldered, etc.) to respective contacts 330 and 332 of the connector 316. FIG. 3 also illustrates that an encapsulant 334 is formed over the other components of the header assembly 302 after the wiring is completed. When installing the header assembly 302 into an implantable device (not shown in FIG. 3), the bottom portions of the conductors 304, 306, 308, and 310 may be routed through an aperture defined by the housing of the implantable device and connected (e.g., wired) to circuitry installed in the housing.

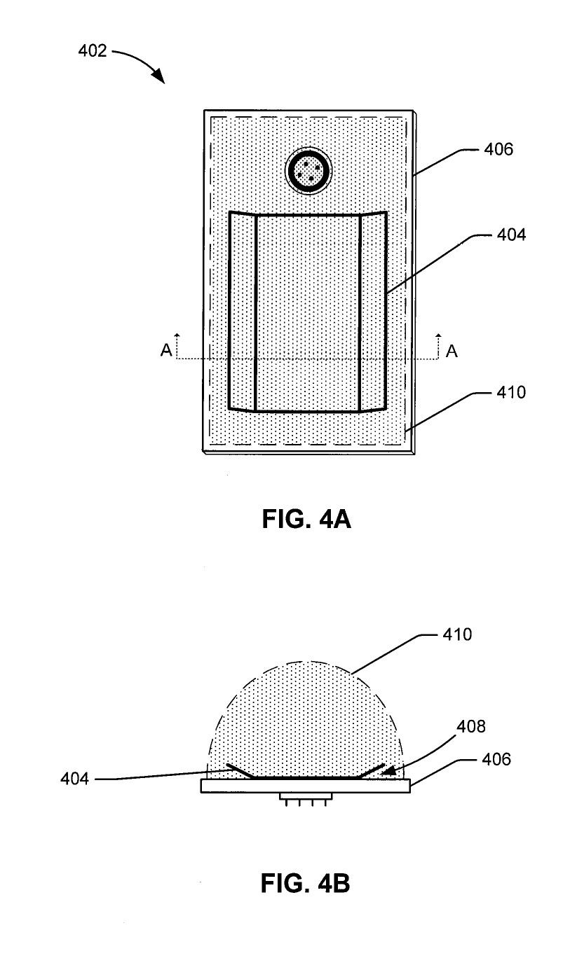

FIGS. 4A and 4B illustrate an embodiment of an anchor member 404 that may be incorporated into (e.g., formed in, or attached to via welding or some other suitable technique) a base 406 of a header assembly 402. As shown in FIG. 4B (corresponding to the view A-A of FIG. 4A), a space 408 is provided under a portion of the anchor member 404. Thus, when an encapsulant 410 is applied to the base 406 during fabrication of the header assembly 402, a portion of the encapsulant 410 will flow into the space 408. Thus, once the encapsulant 410 hardens, it will be mechanically fastened to the base 406 at least in some aspects by the anchor member 404. In addition, the anchor member 404 may protect against differences in coefficient of thermal expansion between the encapsulant 410 and the base 406.

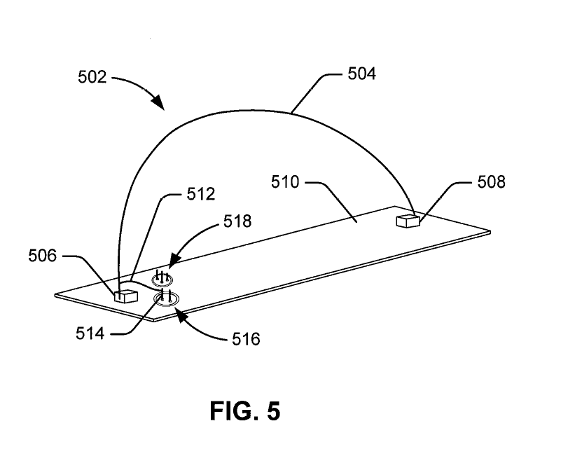

A header assembly may include different types of circuits in different embodiments. FIG. 5 illustrates an embodiment where a header assembly 502 includes an antenna 504 (e.g., for radio frequency telemetry operations). Here, the antenna 504 is fastened to a pair of coupling members 506 and 508 that are attached to a metal base 510 of the header assembly 502. In different implementations one or more of the coupling members 506 and 508 may be insulating or non-insulating, depending on whether the antenna 504 is in an open-loop configuration or a closed-loop configuration. In addition, a conductor 512 couples one end of the antenna 504 to a conductor 514 of a feedthrough 516. It should be appreciated that additional antennas or other circuits (not shown) may be incorporated into a header assembly in a similar manner.

FIG. 5 also illustrates that a header assembly may include more than one feedthrough. For example, the feedthrough 516 of the header assembly 502 may be used to couple signals between the antenna 504 (and, optionally, other circuits of the header assembly 502, not shown) and one or more circuits installed in a housing of an implantable device (not shown in FIG. 5). In addition, a feedthrough 518 may be used to couple signals between other circuits (e.g., one or more connectors, not shown) of the header assembly 502 and one or more circuits installed in the housing.

In some embodiments a header assembly may be fabricated with other components of an implantable device. For example, a header assembly may be prefabricated with a component that is to be placed within a housing of the implantable device.

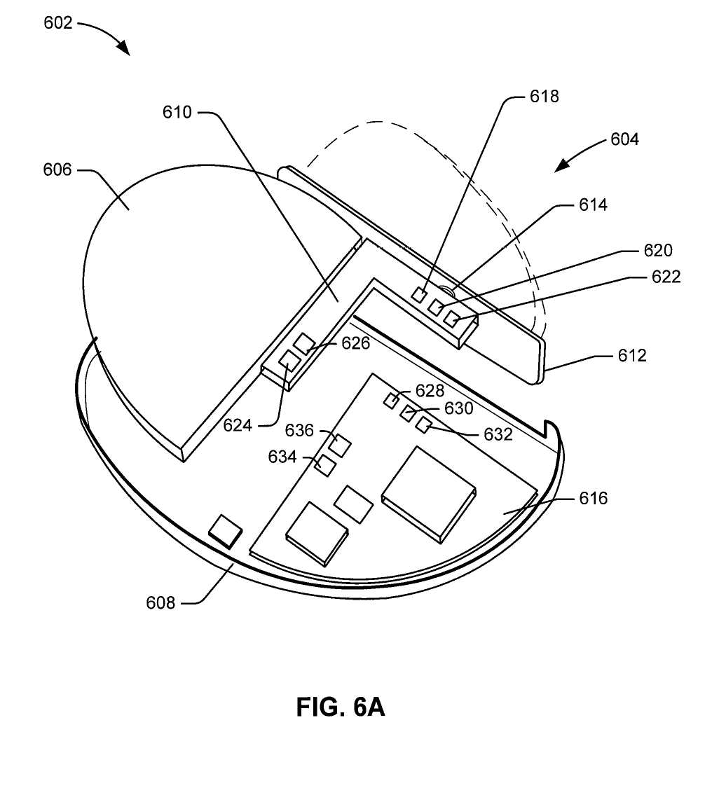

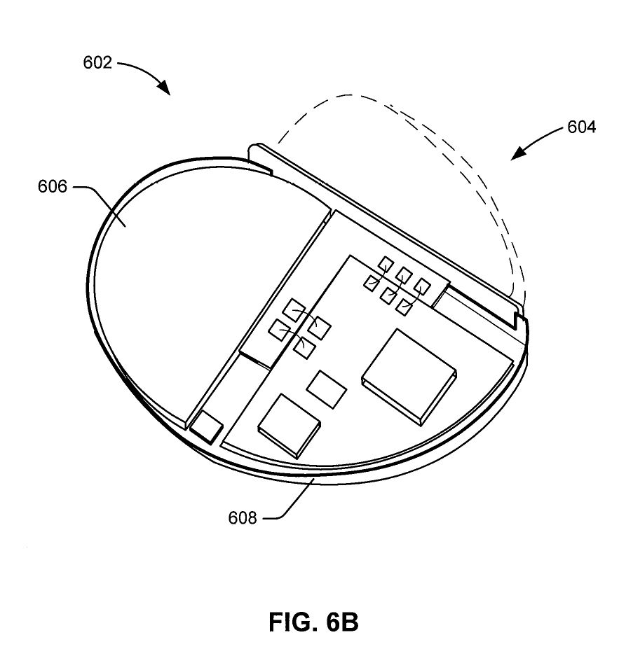

FIGS. 6A and 6B illustrate an embodiment of an implantable device 602 where a header assembly 604 includes a battery assembly 606. Specifically, FIG. 6A is a perspective view showing the header assembly 604 being placed into a bottom portion 608 of a housing of the implantable device 602 and FIG. 6B is a plan view showing the header assembly 604 after it is installed in the bottom portion 608.

Here, a coupling member 610 mechanically couples the battery assembly 606 to a base 612 of the header assembly 604. For example, one portion of the coupling member 610 may be mechanically coupled (e.g., by an attachment mechanism, spring contacts, a solder joint, a weld, a connector, an adhesive, and so on) to the battery assembly 606 and another portion of the coupling member 610 may be mechanically coupled (e.g., as just described) to the base 612 (e.g., to a feedthrough 614 as shown). Alternatively, the coupling member 610 may comprise part of the structure of the battery assembly 606 or the base 612, and is configured to extend to and be coupled with the other component. The coupling member 610 may be constructed of a various materials such as, for example, plastic or metal. In some embodiments the coupling member 610 comprises a flex cable.

The coupling member 610 may include one or more conductors that are coupled to conductors of the feedthrough 614 and/or the battery assembly 606. For example, in the example of FIG. 6A, the coupling member 610 includes several electrical contacts 618, 620, and 622 that are coupled to the conductors of the feedthrough 614 (connection not shown). Similarly, the coupling member 610 includes several electrical contacts 624 and 626 that are coupled to conductors (e.g., battery terminals) of the battery assembly 606 (connection not shown). As described below, the contacts 618-626 may provide an efficient mechanism to couple the conductors of the feedthrough 614 and the battery assembly 606 with conductors of a circuit 616 that is installed in the bottom housing portion 608.

In some embodiments one or more connectors may be used to couple one or more of the contacts 618-626 to conductors of the battery assembly 606 and/or the feedthrough 612. For example, the coupling member 610 may include several plugs that plug into receptacles (not shown) of the battery assembly 606.

Once the header assembly 604 is in place, appropriate connections are made between the contacts 618, 620, 622, 624, and 626 and electrical conductors (e.g., contacts 628, 630, 632, 634, and 636) of the circuit 614. These connections may be implemented in various ways. For example, in some embodiments wires are connected (e.g., using wire bonds, solder, or a weld) to corresponding pairs of contacts as shown in FIG. 6B. In some embodiments one or more flex circuits may be used to couple conductors of the feedthrough 614, the battery assembly 606, and the circuit 616. In some embodiments one or more connectors (e.g., comprising plugs and receptacles) may be used to couple conductors of the feedthrough 614, the battery assembly 606, and the circuit 616.

After the appropriate electrical connections are made between the feedthrough 614, the battery assembly 606, and the circuit 616, a top portion of the housing (e.g., similar to top portion 154 shown in FIG. 1B) may be placed over the bottom housing portion 608. For example, the top and bottom housing portions may be constructed with edges that overlap to some extent and these edges may be slid together at this point. Thus, the implantable device 602 will have one seam between the top and bottom housing portions and another seam between the base 612 and the top and bottom housing portions. All of these seams may then be hermetically welded to provide an implantable device that is hermetically sealed (e.g., as shown in FIG. 1B).

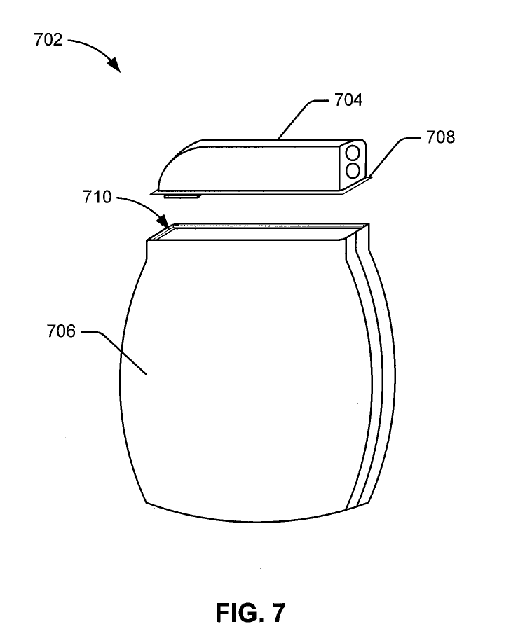

An implantable device as taught herein may take various forms. For example, FIG. 7 illustrates an embodiment of an implantable device 702 where a header assembly 704 attaches to the top of a housing 706. FIG. 7 also illustrates that in some embodiments a base 708 of the header assembly 704 may be placed at least partially within the housing 706 and rest on a ledge 710 (or other suitable structural member) of the housing 706. In such a case, the base 708 is hermetically welded to the housing 706 while being supported by the ledge 710 and inner walls of the top of the housing 706.

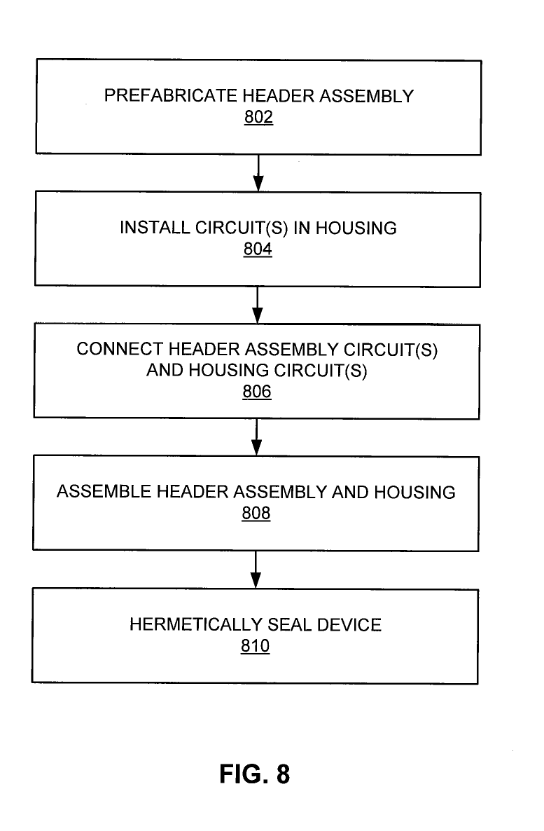

With the above in mind, an embodiment of a process for constructing an implantable device will be described with reference to FIG. 8. For convenience, the operations of FIG. 8 (or any other operations discussed or taught herein) may be described as being performed in conjunction with specific components (e.g., the components of FIGS. 1A and 1B). It should be appreciated, however, that these operations may be performed using different components and/or using a different number of components. It also should be appreciated that one or more of the operations described herein may not be employed in a given implementation.

As represented by block 802 of FIG. 8, a header assembly for the implantable device is prefabricated. For example, connectors for the header assembly may be wired to a feedthrough of a biocompatible metal base. The feedthrough may consist of, for example, a ceramic core that is bonded (e.g., braised) to a biocompatible metal outer ring. This feedthrough may be placed into a hole of the metal base whereupon the outer ring of the feedthrough is hermetically welded to the metal base.

This subassembly may then be placed into a mold whereupon an encapsulant is injected into the mold. As the encapsulant cures, it becomes attached to the metal base (e.g., attached via a chemical bond and/or mechanically fastened). In addition, in embodiments where the header assembly includes a battery assembly, the metal base (e.g., the feedthrough) may be attached to the battery assembly through the use of a suitable coupling mechanism.

As represented by block 804, one or more circuits are installed in the housing of the implantable device. For example, as shown in FIG. 6A a circuit 616 may be placed in a portion 608 of a housing constructed of a biocompatible metal. The particular circuit used here will be designed to meet the requirements of a given application. For example, in an implantable cardiac stimulation device, the circuit may comprise a micro processor, sense circuitry (e.g., for sensing cardiac activity), stimulation circuitry (e.g., for providing cardiac pacing signals and shock pulses), data memory, communication circuitry, and other components.

As represented by block 806, appropriate connections are made between the conductors (e.g., contacts) of the header assembly and corresponding conductors of the circuit that was placed in the housing. For example, as described above in conjunction with FIG. 6B, a connection may be made between contacts of a coupling member and contacts of the housing circuit. Alternatively, in an embodiment that does not employ a coupling member, a similar connection (e.g., using wires, a flex cable, or a connector) may be made between the contacts of a connector (e.g., connector 230 of FIG. 2A) of the header assembly and corresponding contacts of the housing circuit. Similarly, a connection (e.g., using wires, a flex cable, a connector) may be made between contacts of separate battery component and corresponding contacts of the housing circuit. As mentioned above, the electrical connections may be made, for example, using a wire bonding process, a welding process, a soldering process, connectors, or may be made in some other suitable manner.

As represented by block 808, the header assembly and the housing are assembled. Here, the header assembly is positioned over an aperture in the housing (e.g., the edges of the metal base may extend over the edges of the aperture so that the aperture is completely covered). In addition, in a case where the housing consists of multiple subcomponents, these subcomponents are assembled as well. As described above, when the header assembly is placed in the appropriate position relative to the housing, a portion of the feedthrough and/or conductors coupled to the feedthrough may extend through the aperture.

As represented by block 810, the assembled device is hermetically sealed. For example, all of the seams between housing subcomponents and between the header assembly and the housing may be hermetically welded as discussed above.

Various modifications may be incorporated into the disclosed embodiments based on the teachings herein. For example, the structure and functionality taught herein may be incorporated into devices other than the specific types of devices described above. In addition, a housing, a base, and an encapsulant may be made from materials that are different than the materials specifically mentioned above. Also different techniques may be employed to hermetically seal the components of a device together.

The various structures and functions described herein may be incorporated into a variety of apparatuses (e.g., a stimulation device, a monitoring device, etc.) and implemented in a variety of ways. Different embodiments of such an apparatus may include a variety of hardware and software processing components. In some embodiments, hardware components such as processors, controllers, state machines, logic, or some combination of these components, may be used to implement the described components or circuits.

Also, the recited order of the blocks in the processes disclosed herein is simply an example of a suitable approach. Thus, operations associated with such blocks may be rearranged while remaining within the scope of the present disclosure. Similarly, any accompanying method claims present operations in a sample order, and are not necessarily limited to the specific order presented.

In addition, it should be understood that any reference to elements herein using a designation such as “first,” “second,” and so forth does not generally limit the quantity or order of those elements. Rather, these designations may be used herein as a convenient method of distinguishing between two or more different elements or instances of an element. Thus, a reference to first and second elements does not mean that only two elements may be employed there or that the first element must precede the second element in some manner. Also, unless stated otherwise a set of elements may comprise one or more elements.

While certain embodiments have been described above in detail and shown in the accompanying drawings, it is to be understood that such embodiments are merely illustrative of and not restrictive of the teachings herein. In particular, it should be recognized that the teachings herein apply to a wide variety of apparatuses and methods. It will thus be recognized that various modifications may be made to the illustrated embodiments or other embodiments, without departing from the broad scope thereof. In view of the above it will be understood that the teachings herein are intended to cover any changes, adaptations or modifications which are within the scope of the disclosure.

What is claimed is:

1. An implantable device, comprising:
a housing comprising a biocompatible metal and defining an aperture;
a header assembly comprising:
a base constructed of a biocompatible metal and comprising a hermetically sealed feedthrough for at least one conductor, wherein the base is hermetically welded to the housing to cover the aperture;
an encapsulant attached to the base; and
at least one circuit embedded in the encapsulant and coupled to tho at least one conductor; and
at least one other circuit enclosed by the housing and coupled to the at least one conductor.

2. The device of claim 1, wherein the at least one circuit comprises at least one electrical connector.

3. The device of claim 1, wherein the at least one circuit comprises an antenna.

4. The device of claim 1, wherein the header assembly further comprises:
a battery assembly; and
a coupling member configured to mechanically couple the battery assembly to the base.

5. The device of claim 1, wherein the base further comprises an anchor member that mechanically fastens the encapsulant to the base.

6. The device of claim 1, wherein edges of the base extend beyond the aperture such that a bottom surface of the base is positioned upon an outer surface of the housing.

7. The device of claim 1, wherein the encapsulant is attached to the base by at least a chemical bond.

8. The device of claim 1, wherein the encapsulant comprises an epoxy.

9. The device of claim 1, wherein the housing comprises a plurality of subcomponents hermetically welded together.

10. The device of claim 1, wherein the device comprise an implantable cardiac stimulation device.

11. A header apparatus, comprising:
a base constructed of a biocompatible metal and comprising a hermetically sealed feedthrough for at least one conductor;
an encapsulant attached to the base; and
at least one circuit embedded in the encapsulant and coupled to the at least one conductor.

12. The apparatus of claim 11, wherein the at least one circuit comprises at least one electrical connector.

13. The apparatus of claim 11, wherein the at least one circuit comprises an antenna.

14. The apparatus of claim 11, wherein the header assembly further comprises:
a battery assembly; and
a coupling member configured to mechanically couple the battery assembly to the base.

15. The apparatus of claim 11, wherein the base further comprises an anchor member that mechanically fastens the encapsulant to the base.

16. The apparatus of claim 11, wherein the encapsulant is attached to the base by at least a chemical bond.

17. The apparatus of claim 11, wherein the encapsulant comprises an epoxy.
